(19) United States
(12) Patent Application Publication
(10) Pub. No.: US 2026/0283491 A1
(43) Pub. Date: Sep. 24, 2026

(54) DEEP VEIN THROMBOSIS DETECTION DEVICE

(71) Applicant: Scyntek, Inc., Charlotte, NC (US)

(72) Inventors: Karthik RAMAKRISHNAN, Morrisville, NC (US); Daniel Bryson MENICHELLA, Linville, NC (US); Vignesh SRIRAM, Matthews, NC (US); Joshua David HENDERSON, Middleton, WI (US)

(21) Appl. No.: 19/569,985

(22) Filed: Mar. 17, 2026

Related U.S. Application Data
(60) Provisional application No. 63/774,459, filed on Mar. 19, 2025.

(51) Int. Cl. A61B 5/0295; A61B 5/00; A61B 5/02

(52) U.S. Cl. CPC A61B 5/0295; A61B 5/0082; A61B 5/02007; A61B 5/6828; A61B 5/6831; A61B 5/7257; A61B 5/7264; A61B 5/7271; A61B 2560/0462

(57) ABSTRACT

A device having a physiological sensing system configured to analyze a physiological signal using frequency-domain signal processing and embedded on-device screening logic to identify a characteristic associated with a presence or an absence of a venous blood flow abnormality. The physiological sensing system has a biosignal sensor configured to acquire an optical physiological signal from a venous region of a lower limb and a microcontroller configured to execute an algorithm; wherein the biosignal sensor is in signal communication with the microcontroller and the microcontroller uses the algorithm for identification of a feature associated with the presence or the absence of the venous blood flow abnormality; and wherein the device is wearable and non-invasive. The physiological sensing system performs signal acquisition and screening analysis autonomously on the device. Deep vein thrombosis is an example of a venous blood flow abnormality that can be detected by the device.

### CROSS-REFERENCE TO RELATED APPLICATION

This application claims priority from U.S. Provisional Patent Application No. 63/774,459, filed on Mar. 19, 2025, in the United States Patent and Trademark Office. The disclosure of which is incorporated herein by reference in its entirety).

### FIELD OF THE INVENTION

The present invention relates to a device and system for the detection of a potential medical condition, more particularly to a non-invasive device and system for detecting venous blood-flow abnormalities indicative of or associated with the presence or absence of deep vein thrombosis (DVT).

### BACKGROUND OF THE INVENTION

Deep vein thrombosis (DVT) is a serious and potentially fatal condition in which thrombi form in the deep venous system, most commonly in the lower extremities. If untreated, thrombi may travel to the lungs, resulting in pulmonary embolism (PE). Venous thromboembolism (VTE), encompassing DVT and PE, affects up to 900,000 individuals annually in the United States and is responsible for approximately 60,000 to 100,000 deaths each year. A substantial proportion of hospital-associated DVT events occur after patient discharge, with studies estimating that approximately up to 70% of such events develop following discharge from medical hospitalization. During this period, patients typically lack structured clinical monitoring for thrombotic events.

DVT is currently diagnosed and monitored using a combination of clinical risk assessment, laboratory testing, and imaging. The gold-standard diagnostic modality is compression duplex ultrasound, a non-invasive imaging technique that evaluates venous compressibility and blood flow to identify thrombus presence. Commercially available systems and other hospital-based ultrasound platforms provide reliable diagnostic performance when used by trained sonographers. However, duplex ultrasound requires specialized equipment, trained personnel, and a clinical setting. It is operator-dependent and not suitable for frequent, routine, or at-home monitoring. Access is often limited in rural hospitals, outpatient clinics, and community health environments.

Magnetic resonance imaging (MRI) and CT venography may be used in select cases to visualize venous anatomy and thrombus burden. Such systems provide advanced imaging capabilities, but these modalities are costly, require significant infrastructure, and involve long scan times. They are impractical for routine screening or longitudinal monitoring and may be contraindicated in certain patient populations.

D-dimer blood testing is frequently used as a rule-out tool in low-risk patients. Commercial assays measure fibrin degradation products and have high negative predictive value in appropriate populations. However, D-dimer testing lacks specificity, particularly in high-risk groups such as post-surgical patients, oncology patients, pregnant individuals, and hospitalized patients. Elevated D-dimer levels may occur in the absence of thrombosis, leading to false positives and unnecessary imaging. Additionally, D-dimer testing requires an invasive blood draw and specialized equipment to interpret results and does not localize clot location or provide physiologic assessment of venous flow.

Clinical risk scoring systems estimate pretest probability based on symptoms and risk factors. While useful for triage, these scoring systems are indirect assessments and not diagnostic. They rely on subjective clinical inputs and still require confirmatory imaging when risk is moderate or high.

Point-of-care ultrasound (POCUS) systems have expanded access to portable imaging. These devices allow bedside assessment of venous compressibility in emergency or critical care settings. However, POCUS remains operator-dependent and requires training for proper image acquisition and interpretation. Adoption in outpatient, rural, and home-health environments remains limited due to training variability and workflow constraints.

Although effective diagnostic tools exist, they are largely centralized within hospital-based environments and are reactive rather than proactive. Most modalities are episodic; meaning, evaluation occurs only after symptoms develop or when clinical suspicion is high. None are structured for routine, repeatable, at-home physiologic monitoring of venous function.

In high-risk populations including, but not limited to, post-op orthopedic or other surgical patients, oncology patients, general hospitalization patients, pregnant women, individuals with genetic disposition, frequent flyers, immobile individuals, elderly adults, and individuals in rural or resource-limited settings, access to timely imaging is frequently delayed. Because DVT may initially be asymptomatic or minimally symptomatic, reliance on episodic imaging can result in missed opportunities for early detection. Current alternatives for out-of-hospital assessment, including D-dimer testing and clinical risk scoring, do not provide direct physiologic assessment of venous thrombosis and are limited by low specificity in high-risk groups. Advanced imaging modalities are costly and unsuitable for repeated use. Portable ultrasound, while promising, still requires technical expertise and does not eliminate operator dependence.

Despite decades of advancement in imaging and laboratory diagnostics, there remains no widely adopted technology provides structured physiologic monitoring of venous blood flow in a wearable format suitable for home, outpatient, or decentralized care environments that can be conducted by patients themselves.

There is a clear and ongoing need for a non-invasive, low-cost, user-operable technology capable of supporting structured, repeatable venous monitoring in high-risk populations. Such a system would ideally function outside imaging facilities, assist in identifying abnormal venous flow patterns associated with thrombosis or lack thereof, and enable earlier referral for confirmatory diagnostic testing when appropriate.

### SUMMARY OF THE INVENTION

The device and system of the present invention is intended to assist in screening for venous blood-flow abnormalities indicative of or associated with the presence or absence of lower-limb deep vein thrombosis (DVT) and to support decisions regarding the need for confirmatory diagnostic imaging.

In an aspect of the invention, a device comprises: a physiological sensing system configured to analyze a physiological signal using frequency-domain signal processing and embedded on-device screening logic to identify a characteristic associated with a presence or an absence of a venous blood flow abnormality, the physiological sensing system comprising: a biosignal sensor configured to acquire an optical physiological signal from a venous region of a lower limb, a microcontroller configured to execute an algorithm; wherein the biosignal sensor is in signal communication with the microcontroller and the microcontroller uses the algorithm for identification of a feature associated with the presence or the absence of the venous blood flow abnormality; wherein the device is wearable and non-invasive.

In an aspect of the invention, the physiological sensing system performs signal acquisition and screening analysis autonomously, on the device.

In an aspect of the invention, the device supports a form of monitoring selected from the group consisting of vascular, metabolic, systemic, hemodynamic, and a combination thereof.

In an aspect of the invention, the venous blood flow abnormality is deep vein thrombosis.

In an aspect of the invention, the device is selected from one or more wearable form factors.

In an aspect of the invention, the device is in a form of a sleeve or a band.

In an aspect of the invention, the biosignal sensor is a photoplethysmography (PPG) sensor.

In an aspect of the invention, the device comprises: a sleeve configured to be placed on a lower-limb of a person; at least one photoplethysmography (PPG) sensor positioned in the sleeve, wherein the at least one sensor is positioned over a popliteal vein of the lower-limb, a femoral vein of the lower-limb, an iliac vein of the lower-limb, or their associated microvasculature; and a microcontroller positioned in the sleeve, the microcontroller in signal communication with the at least one photoplethysmography (PPG) sensor, wherein the microprocessor is capable of differentiating a blood flow signal indicating an absence of DVT signal and a blood flow signal indicating a potential venous occlusion or an obstruction associated with DVT.

In an aspect of the invention, the microcontroller uses an algorithm for identification of a feature associated with the absence of DVT signal.

In an aspect of the invention, the microcontroller uses an algorithm for identification of a feature associated with a potential venous occlusion or an obstruction associated with DVT.

In an aspect of the invention, the device is non-invasive and/or wearable.

In an aspect of the invention, the device comprises: a band having a detection device unit attached thereto, the band configured to be placed on a lower-limb of a person; at least one photoplethysmography (PPG) sensor attached to the band, wherein at least one sensor is positioned over a popliteal vein of the lower-limb, a femoral vein of the lower-limb, an iliac vein of the lower-limb, or their associated microvasculature; and a microcontroller positioned in the sleeve, the microcontroller in signal communication with the at least one photoplethysmography (PPG) sensor, wherein the microprocessor is capable of differentiating a blood flow signal indicating an absence of DVT signal and a blood flow signal indicating a potential venous occlusion or an obstruction associated with DVT.

In an aspect of the invention, the microcontroller uses an algorithm for identification of a feature associated with the absence of DVT signal.

In an aspect of the invention, the microcontroller uses an algorithm for identification of a feature associated with a potential venous occlusion or an obstruction associated with DVT.

In an aspect of the invention, a method of using a device for determination of deep vein thrombosis is provided. The method comprises: applying the device comprising an optical sensor over a popliteal vein of a lower-limb of a person, a femoral vein of the lower-limb of a person, an iliac vein of the lower-limb of a person, or their associated microvasculature; initiating by the device an automated, timed measurement sequence; acquiring PPG data by the PPG sensor; processing the acquired PPG data by a microcontroller; and indicating whether the acquired physiological signal exhibits a characteristic consistent with a potential venous occlusion or absence thereof.

In an aspect of the invention, processing further comprises performing a frequency-domain characterization of a venous PPG signal.

In an aspect of the invention, the microcontroller uses a feature detection algorithm to identify a feature associated with abnormal blood flow or lack thereof.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is a perspective view of a device having an elastomeric sleeve in accordance with an aspect of the present invention.

FIG. 2 is a side view of the device of FIG. 1 showing the elastomeric sleeve placed on the lower-limb of a person.

FIG. 3 is a perspective view of the device having a band in accordance with an aspect of the present invention.

FIG. 4 is a front view of two bands on the lower-limb of a person.

FIG. 5 is an exploded view of the detection unit/compartment of the device in accordance with an aspect of the present invention.

FIG. 6 a flow chart of the method of operation of the device in accordance with an aspect of the present invention.

FIG. 7 is an example of a process flow diagram where a peak detection algorithm is employed in accordance with an aspect of the present invention.

### DESCRIPTION OF THE PREFERRED EMBODIMENTS

The following description of the embodiments of the present invention is merely exemplary in nature and is in no way intended to limit the invention, its application, or uses. The following description is provided herein solely by way of example for purposes of providing an enabling disclosure of the invention, but does not limit the scope or substance of the invention.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provide illustrative examples for the terms. The meaning of “a,” “an,” and “the” may include plural references, and the meaning of “in” may include “in,” “at,” and/or “on,” unless the context clearly indicates otherwise. The phrase “in one embodiment,” as used herein does not necessarily refer to the same embodiment, although it may.

The terms “Deep Vein Thrombosis” and “DVT”, as used herein generally, refer to formation of a thrombus (blood clot) in deep veins, typically in the lower extremities.

The terms “Photoplethysmography” and “PPG”, as used herein, generally refer to, optical technique measuring changes in blood volume in tissue via light emission, detection, and reflection.

The terms “Venous Refill Time” and “VRT”, as used herein, generally refer to time required for venous blood to refill following compression or elevation.

The terms “Fast Fourier Transform” and “FFT”, as used herein, generally refer to a mathematical transformation that computes the Discrete Fourier Transform, which converts a time-domain signal into a frequency-domain representation. By doing so, one can identify and quantify periodic components of a signal, including dominant frequencies and amplitudes, what may correspond to physiological oscillations (including cardiovascular-associated pulsatile features). In the present invention, FFT is used as a basis of identifying and quantifying abnormalities in obstructed blood flow. The frequency components identified through the FFT analysis may be used to design or configure a frequency-selective filter. The resulting filtered signal may then be analyzed in the time domain to identify oscillatory patterns, waveform characteristics, or other signal features that may be indicative of venous congestion, obstruction, or other abnormalities in blood flow.

The terms “Short-Time Fourier Transform” and “STFT”, as used herein, generally refer to a version of the FFT that operates on a “sliding-window” approach (where each window is the same size) on a time-domain signal and breaks it into segments and then transforms these segments into the frequency domain. This is in contrast to the FFT, which is an average over the entire signal. However, the tradeoff here is that if the window is too small, the temporal information is preserved, but the frequency information is not preserved, while if the window is too large, the frequency information is preserved while the temporal information is not preserved.

The terms “Discrete Wavelet Transform” and “DWT”, as used herein, generally refer to, a type of technique which breaks a time-domain signal into non-sinusoidal oscillations known as “wavelets,” which are bounded (i.e., they start and end at 0) based on a user-defined “mother wavelet” (e.g., including but not limited to, the Daubechies family, the Symlets family, etc.). The “daughter wavelets” are simply compressed and expanded versions of the mother wavelet. Like the STFT, the DWT can provide frequency information with respect to time (i.e., at what time is a frequency most pronounced). However, the main advantage of the DWT is that, unlike the STFT, one can change the window size throughout the signal, so the window size isn't the same for each segment. This is known as Multi-Resolution Analysis.

### Device

The device and system of the present invention are intended to assist in screening for the presence or absence of lower-limb deep vein thrombosis (DVT) and to support decisions regarding the need for confirmatory diagnostic imaging.

The device of the present invention is a compact, non-invasive, and wearable device. Preferably, the device is used for an adult person under clinician oversight. The device of the present invention can take the form of many different form factors including, but not limited to, a band, a sleeve, among others.

The device of the present invention has multi-region or modular sensing architecture. The device supports multiple targeted sensing locations including, for example, configurations in which sensors are positioned over clinically relevant distal and proximal venous regions, including the popliteal, femoral, and iliac venous regions and their associated microvasculature. Signals from these locations may be analyzed independently or in combination to characterize venous flow behavior across multiple anatomical segments. By targeting these regions, the device captures localized hemodynamic behavior rather than relying solely on distal refill dynamics, for example. This distinction changes both the physiological signal origin and the clinical interpretation framework of the measurement. The modular architecture of the device of the present invention thus provides flexibility in sensor placement and enables localized physiological assessment that is not contemplated by existing devices.

The device and system of the present invention use targeted venous-region optical sensing and are optically focused on venous blood flow characterization using PPG, reducing complexity, and maintaining physiological specificity and does not depend upon mechanical or acoustic modalities.

In the device and system of the invention, optical measurement is focused on venous hemodynamics. The device comprises an optical sensing architecture configured to analyze venous hemodynamic signal behavior including, but not limited to, spectral signal characteristics. The device may operate with or without a standardized mechanical maneuver protocol. The analytical focus is therefore on the optical signal behavior itself as opposed to, for example, solely on externally induced waveform morphology.

The device and system are configured to acquire photoplethysmography (PPG) signals from lower extremities of a person and to process the acquired signals using a frequency-domain-based signal processing analysis to identify a physiological pattern(s) indicative of altered venous hemodynamics, thereby enabling screening, detection, monitoring, and/or diagnostic evaluation of venous thrombosis conditions.

It is contemplated and within the scope of the invention that the detection device may also implement signal normalization strategies (e.g., baseline correction, adaptive filtering, etc.) to reduce patient-to-patient variability.

The device and system perform frequency-domain characterization of venous PPG signals. Acquired signals may be transformed into a spectral representation using techniques such as Fourier transforms or related spectral decomposition methods. The resulting frequency components allow identification of repeatable spectral patterns associated with altered venous hemodynamics. For example, experimental observations indicate a repeatable increase in spectral energy within a defined frequency range under simulated venous occlusion conditions. These spectral characteristics provide an additional analytical dimension not used in existing methods.

In regard to frequency-domain characterization of venous flow signals as a primary analytical component, the detection device of the present invention applies a method(s) using spectral decomposition techniques to characterize frequency-domain components of the venous PPG signal. It evaluates repeatable spectral energy distributions associated with altered venous return patterns, including features observed in the 0.6 to 1.0 Hz range under simulated occlusion conditions. Unlike conventional approaches that apply filtering or smoothing primarily for noise reduction, the frequency-domain characterization in the present invention is applied to facilitate identification of repeatable spectral features associated with altered venous return, thereby enhancing the detection and analysis of various hemodynamics patterns and features relevant to DVT screening. In addition, the frequency-based characterization allows for stronger time-domain characterization through features such as peak detection.

It is contemplated and within the scope of the invention that the detection device may utilize other signal processing methods, such as integrating statistical methods, machine learning techniques, or other pattern recognition algorithms for finding hidden patterns within the time domain and/or frequency domain in DVT-associated flow that are not present in non-DVT-associated flow or removing noise from other factors (such as involuntary movement).

The device of the present invention uses embedded on-device signal processing and decision logic. The device performs digital filtering, spectral analysis, and screening decision logic directly on an embedded microcontroller within the wearable device itself. This supports autonomous operation (i.e., one where the patient can operate the device by themselves), consistent performance, and reduced dependency on connectivity or external software environments. This architecture also allows the device to generate screening outputs locally without requiring external computing resources for primary interpretation. While external software may optionally be used for visualization or record-keeping, the core data analysis is capable of operating autonomously on the device. Thus, the device of the present invention provides a compact wearable sensing platform with embedded signal processing, allowing autonomous operation outside specialized vascular laboratories.

### Method of Use

In accordance with an aspect of the invention, a method of using the device is provided.

The method of use of the device comprises: applying the device to a lower-limb of a person; initiating by the device an automated, timed measurement sequence; acquiring PPG data by the PPG sensor; processing the acquired PPG data by a microcontroller; and generating an output indicative of a venous blood-flow assessment, the output being provided on the device, with options to be transmitted to an external system, or otherwise communicated to a user or clinician.

During the initiated sequence, a person may remain at rest or perform a standardized physiologic maneuver (such as, but not limited to, ankle flexion or leg elevation). After the optional generation of a structured clinician-facing report, if absence of DVT is not indicated, the person may be referred for further testing such as a duplex ultrasound, for example. The detection device of the invention is suitable for repeatable monitoring during elevated thrombotic risk periods, such as a post-operative window of time.

### End-Use Applications of Device

The detection device of the invention may be used for structured venous assessment, screening, or monitoring in any number of settings, including but not limited to, clinical, outpatient, bedside, or home settings. The device is configured for simplified, operator-independent use and may be deployed by healthcare professionals, caregivers, or patients under or without clinician oversight. In certain implementations, the device may generate outputs derived from photoplethysmography signals to support evaluation of venous blood-flow characteristics, including indicators such as venous refill behavior, hemodynamic trends, or other physiological features associated with venous abnormalities, thereby enabling structured surveillance of individuals at elevated risk of venous thrombotic conditions.

The device of the invention may be used in, but not limited to, post-surgical recovery monitoring (orthopedic, oncologic, general surgery), oncology patients with elevated thrombotic risk, military personnel or veterans with elevated thrombotic risk, elderly and immobile patients, rural or access-limited populations, skilled nursing and rehabilitation facilities, hospital discharge monitoring programs, general in-hospital monitoring for at-risk patients, among others.

The device enables structured, repeatable venous assessment outside of imaging laboratories and without the need for Doppler ultrasound or trained sonographers. It supports earlier identification of patients requiring confirmatory duplex ultrasound and may reduce unnecessary imaging when absence is indicated.

In certain implementations, the device may interface with external computing systems or monitoring platforms to support remote or longitudinal assessment of venous blood-flow characteristics. Such systems may enable review of device-generated outputs by clinicians or caregivers and may support monitoring over extended periods of elevated thrombotic risk. Applications may include, but are not limited to, post-operative monitoring, surveillance in oncology patients or individuals with hypercoagulable conditions, or other clinical scenarios where repeat venous assessment may be beneficial.

The device of the invention can be used for structured venous assessment, providing outputs beyond simple binary screening for the presence or absence of DVT. In certain embodiments, the device may support venous mapping, evaluation of venous return dynamics, or a combination of such assessments.

The device of the invention can be used for operator-independent bedside and home screening. One-button operation and embedded firmware of the detection device allow use by nurses, medical assistants, patients in any setting (under or without clinician oversight), caregivers in low-resource settings, among others.

Immediate public health applications include, but are not limited to, reducing unnecessary hospital readmissions, extending DVT surveillance beyond inpatient settings, supporting decentralized care models, and increasing access to vascular screening in rural environments.

Regarding structured clinician-facing output and longitudinal trend capability, the device of the present invention produces structured outputs designed for clinician interpretation and documentation. This facilitates integration into physician-supervised monitoring programs and supports repeated trend review during high-risk periods such as post-operative recovery.

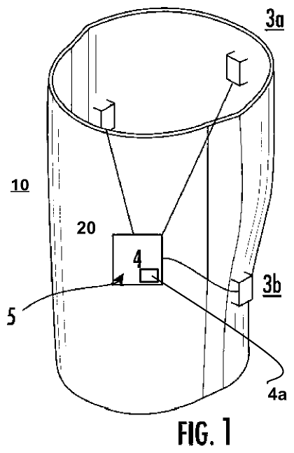

Referring now to the figures, FIG. 1 is a perspective view of a device 10 having a sleeve 20 in accordance with an aspect of the present invention. The sleeve can be an elastomeric sleeve. As shown in FIG. 1, device 10 is a non-invasive, wearable device. Sleeve 20 is configured to fit over a lower-limb of a person such as a knee and/or a thigh. Embedded in the sleeve are biosignal sensors 3a and 3b (such as PPG sensors) which are to be positioned over a venous region such as a femoral vein, a popliteal vein, and/or an iliac vein, or their associated microvascular, when the sleeve is placed on the person. Also embedded within sleeve 20 is a microcontroller 4. The term “microcontroller”, as used herein, generally refers to a type of microprocessor that contains a CPU/processor, memory, and input/output (I/O) peripherals.

Microcontroller 4 houses an algorithm 4a configured to identify a presence or an absence of an abnormality associated with obstructed blood flow (such as within DVT) as opposed to physiologically healthy blood flow (such as a non-DVT case).

In accordance with FIG. 1, microcontroller 4 receives a signal(s) from biosignal sensors 3a and 3b configured for venous-region optical sensing to determine DVT signal versus non-DVT signal. Microcontroller 4, as shown in FIG. 1, has an on-board alert system 5 for indication of detection of a potential abnormality. The alert system can take the form of a red/green LED or a buzzer system or other notification to provide a visual signal and/or auditory signal of the possibility of DVT or lack thereof. Such alert system can also be used to let the user know when the limits are exceeded such as when the test is over or when there is a notification of results, for example.

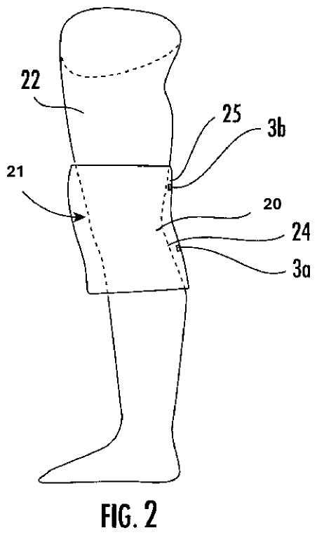

FIG. 2 is a side view of a lower-limb 22 of a person with sleeve 20 positioned over a knee 21 such that a first biosignal sensor 3a is positioned over a popliteal vein or its associated microvasculature 24 and a second biosignal sensor 3b is positioned over a femoral vein or its associated microvasculature 25.

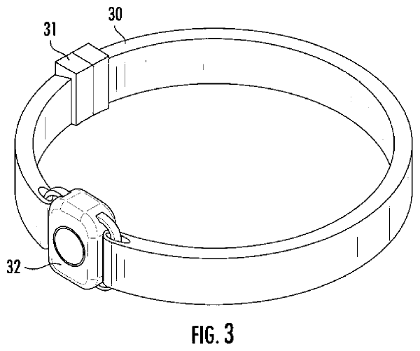

In another aspect of the invention, the device is in a form of a band. As shown in FIG. 3, a band 30 is configured for attachment to a leg. Band 30 is configured to fit on the lower-limb of a person over the popliteal vein, the femoral vein, the iliac vein, or their associated microvasculature. If one or more veins are to be covered, two bands are used. Band 30 is designed to fit over the calf, knee or thigh, and band 30 has a closure 31 for placing the band around the leg. Band 30 has a detection unit 32 which comprises a PPG biosignal sensor among other components.

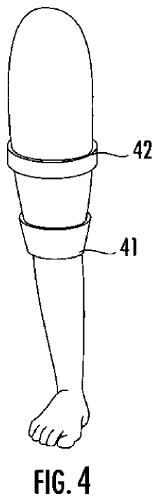

FIG. 4 is a view of a lower-limb of a person with a first band and a second band 41 at a location for venous-region optical sensing.

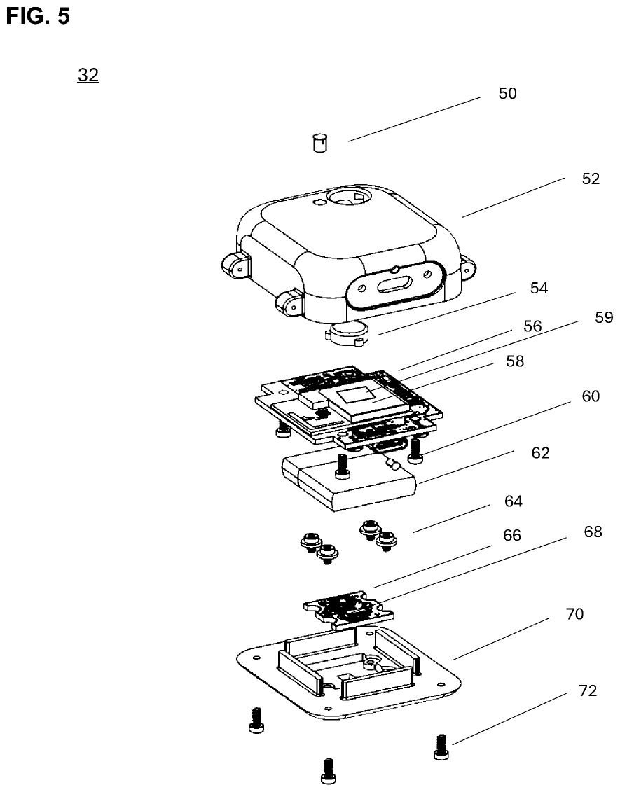

FIG. 5 is an exploded view of detection unit 32 of the device in accordance with an aspect of the present invention. As shown in FIG. 5, the detection unit comprises an upper enclosure 52 and a bottom enclosure 70. A light pipe 50 can be inserted in upper enclosure 52 as a visual indicator. A button 54 can also be inserted in upper enclosure 52 for activation of the device by the user.

A microcontroller 58 is present on a main printed circuit board (PCB) 56. PCB mounting screws 60 are used to secure the PCB 56 within the detection unit. A battery 62 is enclosed within the detection unit for use in activation of the detection device. The detection unit 32 is battery-powered and optimized for stable skin coupling, reduced motion artifact, ambient light rejection, and/or repeatable anatomical positioning.

Daughterboard mounting screws 64 (with washers) are shown for securing a Daughterboard 66 containing a biosignal sensor with optical front end 68 within the detection unit. Additional screws or fasteners 72 are shown to secure bottom enclosure 70 to upper enclosure 52. Non-limiting examples of biosignal sensors include, but are not limited to, photoplethysmography (PPG) sensors having an optical front-end, integrated LED drivers, photodiode amplification, and Analog-to-Digital (ADC) components. Non-limiting examples of commercially available PPG biosignal sensors include, but are not limited to, MAX30101 commercially available from Analog Devices, AFE4404 from Texas Instruments, Si1144 from Silicon Labs, among others.

The PPG sensor(s) measure variations in light absorption caused by volumetric blood changes within tissue. Longer wavelength light (e.g., near-infrared) penetrates deeper tissue and is suitable for interrogation of venous structures. Reflected light is captured by photodetectors and converted to analog electrical signals. An “optical front end” typically comprises a LED, LED driver, photodiode, transimpedance amplifier, analog filtering, and Analog-to-Digital (ADC) converter that allows for data to be read by the microcontroller. The PPG sensor creates a measurable electrical signal from light that can be used to assess an abnormality (or lack thereof) in blood flow that can be distinguished from physiologically heathy blood flow.

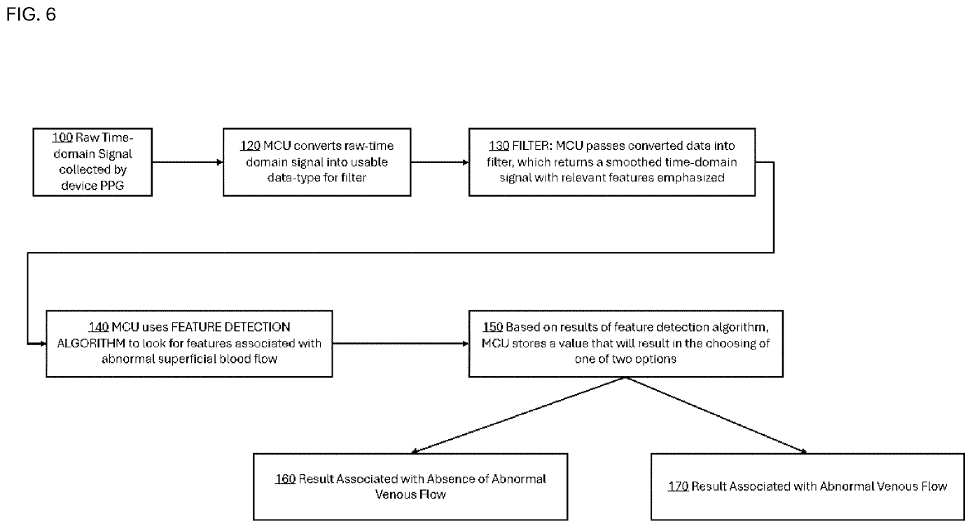

FIG. 6 is a flow chart of a method of operation of the device in accordance with an aspect of the invention. Referring to FIG. 6, in step 100, the optical front end of the PPG sensor collects a raw-time domain signal for a period of time, such as approximately 60 seconds to 90 seconds, from three different wavelengths (red, green, and IR). Each LED within the sensor that generates the corresponding wavelength is embedded within the sensor itself and sends or transmits the signal data to the microcontroller (MCU).

In step 120, the microcontroller on board converts this data from a time domain into a frequency domain with the FFT. The frequency domain allows for the periodicity of the blood flow the device is detecting to be more closely analyzed via plotting the signal over the dominant frequencies present within it and quantify how the abnormalities in blood flow are affecting the expected periodic behavior of blood flow. In step 130, a digital filter highlighting abnormalities located at certain frequencies can then be applied to the time-domain signal, resulting in a new time-domain signal that accentuates abnormalities characterized by certain features (for example, including, but not limited to, higher peaks, faster rise times, etc.) that are either not present or significantly reduced in physiologically healthy signals. In step 140, time-domain methods and feature detection algorithm (for example, including, but not limited to, peak detection) can then be applied to the smoothed signal to determine if there are features indicative of abnormal venous blood-flow patterns present. Based on the results of the feature detection algorithm, in step 150, MCU stores a value that will result in the choosing of one of two options. In step 170, if one or more features indicative of abnormal venous flow are detected, the device may generate an alert, for example, by activating a visual indicator including, but not limited to, a red LED or an auditory indicator such as an alarm. In step 160, if such features are not detected, the device may generate a different type of indicator including, but not limited to, a green LED or other confirmation. In certain embodiments, features associated with physiologically healthy blood flow may also be detected and mapped to a corresponding indicator. The particular assignment of features to output signals is not limited to any specific mapping.

Unlike the FFT, which provides a single average frequency spectrum for the entire signal rather than key, time-localized frequency components, physiological events such as blood flow changes due to DVT are often transient and non-stationary, meaning that their unique frequency signatures may only be present for a short duration, appearing as a spike in the time domain. To build more effective filters, analyses may include Short-Time Fourier Transform (STFT) or Discrete Wavelet Transform (DWT), which may be used in conjunction or in place of the FFT. These methods (the STFT and DWT) are more computationally intensive but can provide improved temporal localization of frequency components, enabling identification of when particular frequencies or frequency abnormalities occur within the signal over time.

The microcontroller uses a feature detection algorithm (shown as 59 in FIG. 5) to look for features associated with abnormal superficial blood flow. Non-limiting examples of features subject of the algorithm include, but are not limited to, peak detection, zero crossing rate, signal energy, signal envelope detection, signal peak width, spectral feature tracking, signal derivative/slope detection, signal pulse area, signal perfusion index, venous refill time, and a combination thereof.

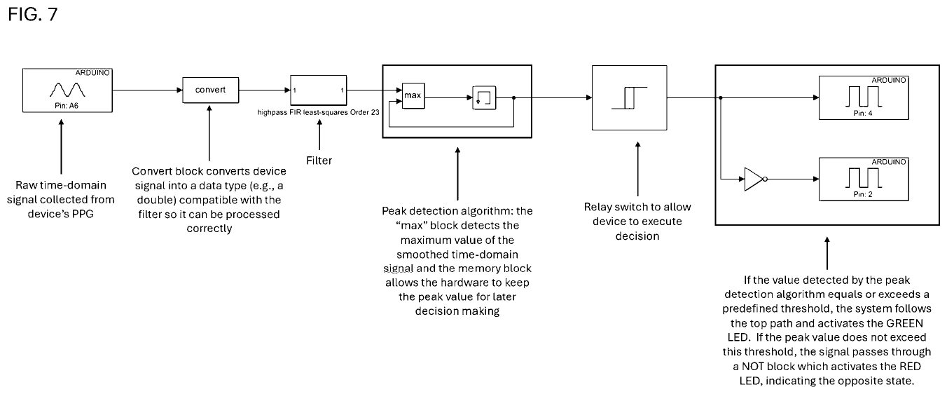

FIG. 7 is an example of a process flow diagram where a peak detection algorithm is employed in accordance with an aspect of the present invention. FIG. 7 illustrates the signal processing pipeline from raw time-domain signal to hardware making a decision on which light to turn on after domain transformation, filtering, and feature detection. As shown in FIG. 7, raw-time domain signal is collected from the PPG sensor of the device. A convert block converts device signal into a data type (e.g., double) compatible with the filter so it can be processed correctly. As illustrated, the filter implements a Highpass FIR least-squares order 23 methodology. In the Peak detection algorithm, the “max” block detects the maximum value of the smoothed time-domain signal and the memory block allows the hardware to keep the peak value for later decision-making. The relay switch allows the device to execute the decision. If the value detected by the peak detection algorithm equals or exceeds a predefined threshold, the system follows the top path and activates the GREEN LED. If the peak value does not exceed this threshold, the signal passes through a NOT block which activates the RED LED, indicating the opposite state.

### EXAMPLES

Experimental data from small-scale pre-clinical testing conducted with healthy volunteers under simulated DVT conditions using controlled venous occlusion (manual occlusion or cuff-based venous outflow restriction) was obtained.

In these experiments, PPG signals were collected from the lower limb of a person using a wearable prototype of the device positioned over regions associated with venous return. Venous occlusion conditions created transient, reversible venous flow alterations intended to approximate selected hemodynamic features consistent with impaired venous return. The signal processing of the detection device demonstrated the ability to distinguish occlusion-associated signal patterns from non-occluded baseline conditions. Representative plots from testing showed visible differences between non-occluded and occluded conditions in venous refill behavior and waveform structure, supporting feasibility of physiologic differentiation are described herein below.

### PPG Signals—No Simulated DVT vs Simulated DVT

Tests were conducted that provided representations of DVT signals and non-DVT signals. Two signals, the venous refill time (VRT) of simulated DVT and no simulation (physiologically healthy) DVT, were obtained from a commercial MAXREFDES103 PPG sensor from Analog Devices. This simulation was done by occluding the popliteal vein and then having a participant perform calf exercise via dorsal flexion to increase intracellular pressure and force blood out of the leg and to the heart. The results were graphically plotted. The x-axis was time (in seconds), and the y-axis was the amplitude of the PPG signal. VRT was a metric used to assess how long it takes for blood to return to the veins. In the simulated DVT case, there was no noticeable VRT (assessed visually by comparing the peak of the signal to the baseline value which was indicated by the settling value of the signal) in comparison to the physiologically healthy case. As such, this indicated that by using PPG, one could see the difference between DVT and non-DVT.

### DVT vs No DVT

The signals present here were the results of occluding the top of the popliteal vein with a blocker to better simulate the effects of blood flow after DVT. Participants were asked to perform dorsiflexion exercises in both cases (one with the blocked popliteal vein, the other without the blocked popliteal vein). The x-axis was time (in seconds) and the y-axis was the amplitude of the PPG signal. The DVT signals were characterized by large amplitude changes from their baseline compared to their non-DVT counterparts. These larger amplitude changes reflect abrupt changes in venous blood flow volume caused by the simulated blockage.

### Logarithmic Power Spectrum of DVT vs Non-DVT

The signals present here were the power spectrum frequency-domain representation of the signals in the above discusses graph. The x-axis was frequency (in Hz) and the y-axis was the logarithmic amplitude of PPG frequency signal. All of the DVT signals had a fiducial mark (significant elevation of amplitude) at 1 Hz while all of the non-DVT signals did not have this fiducial mark. This hump was used to build a filter to isolate relevant features within the time domain.

The resulting time domain signal, obtained by passing the raw time domain signal through the frequency-domain-derived filter built from the fiducial mark, exhibited accentuated features indicative of altered venous hemodynamics and was suitable for subsequent analysis. A graphical representation was plotted in which the x-axis was time (in seconds) and the y-axis was the amplitude of the PPG signal. The fluctuations present at ~7-15 s, ~23-30 s, and ~35-42 s were all present only within the DVT signals. These fluctuations were not present within the non-DVT signals. This information was used to tune the algorithmic model to look for differences like these.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A device comprising:
a physiological sensing system configured to analyze a physiological signal using frequency-domain signal processing and embedded on-device screening logic to identify a characteristic associated with a presence or an absence of a venous blood flow abnormality, the physiological sensing system comprising:
a biosignal sensor configured to acquire an optical physiological signal from a venous region of a lower limb,
a microcontroller configured to execute an algorithm;
wherein the biosignal sensor is in signal communication with the microcontroller and the microcontroller uses the algorithm for identification of a feature associated with the presence or the absence of the venous blood flow abnormality;
and wherein the device is wearable and non-invasive.

2. The device according to claim 1, wherein the physiological sensing system performs signal acquisition and screening analysis autonomously, on the device.

3. The device according to claim 1, wherein the device supports a form of monitoring selected from the group consisting of vascular, metabolic, systemic, hemodynamic, and a combination thereof.

4. The device according to claim 1, wherein the venous blood flow abnormality is deep vein thrombosis.

5. The device according to claim 1, wherein the device is configured in one or more wearable form factors.

6. The device according to claim 1, wherein the device is in a form of a sleeve or a band.

7. The device according to claim 1, wherein the biosignal sensor is a photoplethysmography (PPG) sensor.

8. A device for detection of deep vein thrombosis (DVT), the device comprising:
a sleeve configured to be placed on a lower-limb of a person;
at least one photoplethysmography (PPG) sensor positioned in the sleeve, wherein the at least one sensor is positioned over a popliteal vein of the lower-limb, a femoral vein of the lower-limb, an iliac vein of the lower-limb, or their associated microvasculature; and
a microcontroller positioned in the sleeve, the microcontroller in signal communication with the at least one photoplethysmography (PPG) sensor, wherein the microprocessor is capable of differentiating a blood flow signal indicating an absence of DVT signal and a blood flow signal indicating a potential venous occlusion or an obstruction associated with DVT.

9. The device according to claim 8, wherein the microcontroller uses an algorithm for identification of a feature associated with the absence of DVT signal.

10. The device according to claim 8, wherein the microcontroller uses an algorithm for identification of a feature associated with a potential venous occlusion or an obstruction associated with DVT.

11. The device according to claim 8, wherein the device is non-invasive.

12. The device according to claim 8, wherein the device is wearable.

13. A device for detection of deep vein thrombosis (DVT), the device comprising:
a band having a detection device unit attached thereto, the band configured to be placed on a lower-limb of a person;
at least one photoplethysmography (PPG) sensor attached to the band, wherein at least one sensor is positioned over a popliteal vein of the lower-limb, a femoral vein of the lower-limb, an iliac vein of the lower-limb, or their associated microvasculature; and
a microcontroller positioned in the sleeve, the microcontroller in signal communication with the at least one photoplethysmography (PPG) sensor, wherein the microprocessor is capable of differentiating a blood flow signal indicating an absence of DVT signal and a blood flow signal indicating a potential venous occlusion or an obstruction associated with DVT.

14. The device according to claim 13, wherein the microcontroller uses an algorithm for identification of a feature associated with the absence of DVT signal.

15. The device according to claim 13, wherein the microcontroller uses an algorithm for identification of a feature associated with a potential venous occlusion or an obstruction associated with DVT.

16. The device according to claim 13, wherein the device is non-invasive.

17. The device according to claim 13, wherein the device is wearable.

18. A method of using a device for determination of deep vein thrombosis, the method comprising:
applying the device comprising an optical sensor over a popliteal vein of a lower-limb of a person, a femoral vein of the lower-limb of a person, an iliac vein of the lower-limb of a person, or their associated microvasculature;
initiating by the device an automated, timed measurement sequence;
acquiring PPG data by the PPG sensor;
processing the acquired PPG data by a microcontroller; and
indicating whether the acquired physiological signal exhibits a characteristic consistent with a potential venous occlusion or absence thereof.

19. The method according to claim 18, wherein processing further comprises performing a frequency-domain characterization of a venous PPG signal.

20. The method according to claim 18, wherein the microcontroller uses a feature detection algorithm to identify a feature associated with abnormal blood flow or lack thereof.

21. The method according to claim 18, wherein the device is non-invasive.

22. The method according to claim 18, wherein the device is wearable.
